(19) United States
(12) Patent Application Publication
(10) Pub. No.: US 2015/0343102 A1
(43) Pub. Date: Dec. 3, 2015

(54) PHOTOCHROMIC DETECTION OF ULTRAVIOLET IRRADIATION

(71) Applicant: PurpleSun Inc., New York, NY (US)

(72) Inventors: Luis F. Romo, New York, NY (US); Matthew John Heidecker, Waynesville, OH (US)

(21) Appl. No.: 14/724,653

(22) Filed: May 28, 2015

Related U.S. Application Data
(60) Provisional application No. 62/004,008, filed on May 28, 2014.

(51) Int. Cl. A61L 2/10; A61L 2/28; A61L 9/20; G01N 31/22

(52) U.S. Cl. CPC A61L 2/10; G01N 31/226; A61L 2/28; A61L 9/20

(57) ABSTRACT

The present invention provides methods for detecting electromagnetic energy (e.g., ultraviolet radiation) and articles suitable for use in such methods. The methods and articles can be useful for detecting sterilization or disinfection resulting from electromagnetic energy such as ultraviolet C. Detection of the sterilization or disinfection allows discontinuation of the exposure when a desired level of sterilization or disinfection has been obtained. Feedback mechanisms for controlling exposure to the energy may also be used.

### CROSS-REFERENCE

The present application is non-provisional of, and claims the benefit of U.S. Provisional Patent Application No. 62/004,008 (Attorney Docket No. 46747-705.101) filed on May 28, 2014; the entire contents of which are incorporated herein by reference.

### BACKGROUND OF THE INVENTION

Microbial contamination is a global concern within many industries such as the healthcare industry and food industry. It costs countries billions of dollars in expenses per year, and, more importantly, the contaminant pathogens plague private and public (e.g. healthcare) settings and surroundings. Ultimately, these contaminated surroundings lead to infections and can ultimately lead to death. For example, the United States Center for Disease Control (CDC) encourages hospitals to develop programs to optimize cleaning protocols for high-touch articles and surfaces as part of room cleanings at the time of discharge or transfer of patients due to evidence that the transmission of many healthcare acquired pathogens is related to contamination of such surfaces. See, e.g., Weber et al, Am. J. Infect. Control, 38:S25-33, 2010.

The art of sterilization and disinfection utilizing ultraviolet C (UV-C) light energy is a widely accepted and known process. Many technologies have been developed for application of UV-C light energy for water, air, and surface disinfection and sterilization. The only current measurement solutions to date, utilize UV-C specific electromechanical meters to quantify light intensity and that are programmed to detect UV-C wavelengths. These meters are expensive and only provide numerical values of UV-C intensity. To date, this is the only tool for measuring levels of UV-C sterilization and disinfection. Film or badge indicators have been used in many other applications outside of UV-C disinfection and sterilization such as autoclave sterilization tape, radiology badge, pH testing and more. Never has the UV-C industry benefited from an indicator to quickly communicate the effectiveness of UV-C disinfection or sterilization on specific articles and surfaces that benefit from the sterilization or disinfection.

Therefore, there has been no satisfactory way for a user to easily perceive the sterilization or disinfection of articles or areas (e.g., a surface, an instrument, etc.) after exposure to ultraviolet (UV) light. As such, it has been very difficult at best for a user to tell when an article has been thoroughly sterilized or disinfected or whether continued exposure is required. Accordingly, methods and articles for detecting the sterilization or disinfection of an article or area via UV light are disclosed herein. Optionally, provided methods and/or indicator elements detect the degree of sterilization or disinfection of the articles or area being sterilized or disinfected. For at least these reasons, there is need for improved devices and methods which indicate when sterilization or disinfection has been achieved. At least some of these objectives will be met by the disclosure provided below.

### SUMMARY OF THE INVENTION

The invention may involve the use of an indicator element such as photochromic molecules or polymers incorporating a photochromic molecule. Photochromic molecules have been heavily explored for applications related to UVA and UVB involving consumer toys, visual lenses and others. Never before have these materials been utilized for microbiological use involving UV-C safety, disinfection and sterilization applications.

Optionally, the invention provides methods of detecting electromagnetic energy (e.g., ultraviolet radiation) comprising the use of indicator elements having one or more photochromic surfaces or other materials that react to particular wavelengths of electromagnetic energy. The invention also optionally provides indicator elements that may be used in such methods.

Optionally, a method may include the following steps:

(a) providing an indicator element comprising a photochromic surface or material;

(b) exposing said indicator element to electromagnetic energy having a wavelength preferably between about 100 nm and about 290 nm, or more preferably between about 200 nm-280 nm, or even more preferably between about 240 nm-270 nm where the exposure results in one or more visual and/or textural changes to said photochromic surface or material that can be seen or felt, or otherwise detected;

(c) detecting the one or more visual and/or textural changes of said indicator element which may comprise one or more photochromic surfaces or one or more materials; and

(d) terminating the ultraviolet radiation exposure after the visual and/or textural changes are detected and consistent with sterilization or disinfection of the photochromic surface or the material to a desired degree or level in correlation with the dosage of ultraviolet radiation received by the indicator element or an adjacent article being sterilized or disinfected.

Optionally, the visual and/or textural changes are reversible. Optionally, the visual and/or textural changes are irreversible.

Optionally, the electromagnetic energy comprises light with a wavelength of between about 100 nm to about 120 nm, about 120 nm to about 140 nm, about 140 nm to about 160 nm, about 160 nm to about 180 nm, about 180 nm to about 200 nm, about 200 nm to about 220 nm, about 220 nm to about 240 nm, about 240 nm to about 260 nm, or about 260 nm to about 280 nm. Optionally, the electromagnetic energy comprises light with a wavelength between about 240 nm and 260 nm. Optionally, the electromagnetic energy comprises light with a wavelength of about 254 nm.

Optionally, the electromagnetic energy is provided by a source (e.g., a UV light source) with an output between about 100 and 1,000 watts (W). Optionally, the source provides an output of about 800 W.

Optionally, electromagnetic energy is provided at a dose between about 500 to about 450,000 μW-sec/cm². Optionally, electromagnetic energy is provided at a dose between about 500 to about 55,000 μW-sec/cm².

According to various options, the length of time for which an article or area is exposed to electromagnetic energy may vary according to the specific requirements of a particular application. Optionally, an article or area in which sterilization or disinfection is desired is exposed to electromagnetic energy for between about 0.01 seconds to about 180 seconds. Optionally, the exposure is for between about 10 seconds and about 120 seconds.

Optionally, the article or area to be sterilized or disinfected is between about 0.5 feet to about 12 feet away from a source of electromagnetic energy (e.g., a UV light source).

Optionally, a variety of tailored materials may be used to indicate the sterilization or disinfection. Optionally, a tailored material comprises a photochromic monomer, a photochromic oligomer, or a photochromic polymer containing a photochromic molecule (e.g. chemically bonded together or blended together). Other indicator elements may be used and therefore the present invention is not limited to the use of photochromic materials. Any material which reacts to and indicates exposure to ultraviolet light may be used.

Optionally, a photochromic surface comprises a photochrome that comprises a photochromic small molecule having a molecular weight of less than 1,000 Daltons. Optionally, a photochromic small molecule may be a pyran or a spiropyran such as benzo[3,4]fluoreno[2,1-b]pyran-13-ol, 3,13-dihydro-3,3-bis(4-methoxyphenyl)-6,11,13-trimethyl-,

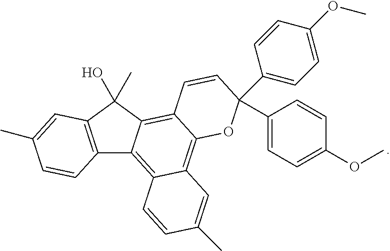

Optionally, an indicator element such as a photochromic surface may comprise a photochrome comprising an optionally substituted coumarin moiety, an optionally substituted spirooxazine moiety, an optionally substituted naphthoaxazine moiety, an optionally substituted naphthopyran moiety, an optionally substituted phenoxyanthraquinone moiety, an optionally substituted carbazole moiety, an optionally substituted spiropyran moiety, or combinations thereof. Optionally, the photochromic surface may be fixedly or releasably coupled to the article being sterilized or disinfected.

Optionally, sterilization or disinfection may be partial or complete sterilization or disinfection of a surface, article, or area. Optionally, sterilization or disinfection comprises at least one of air disinfection, water disinfection, and surface disinfection.

Optionally, sterilization or disinfection may be identified and/or characterized by a microbial kill efficacy. Optionally, an article, area, or surface may be considered sterilized or disinfected if exposure to electromagnetic energy achieves a microbial kill efficacy of at least 85%, at least 88%, at least 90%, at least 91%, at least 92%, at least 93%, at least 94%, at least 95%, at least 96%, at least 97%, at least 98%, at least 99%, at least 99.2%, at least 99.5%, or at least 99.9%, or at least 99.9999%.

Optionally, an article, area or surface may be considered sterilized or disinfected if exposure to electromagnetic energy is sufficient to inactivate and/or kill at least one of a bacteria, a mold, a protozoa, a virus, a yeast, a spore, or any combination thereof.

In an aspect of the present invention, a method of sterilizing or disinfecting an article comprises providing an indicator element adjacent the article, wherein the indicator element reacts to and indicates exposure to electromagnetic energy, and exposing the indicator element and the article to electromagnetic energy having a wavelength between about 100 nm and about 280 nm, wherein the exposure results in one or more visual and/or textural changes to the indicator element. The method also may comprise detecting the one or more visual and/or textural changes of the indicator element; and terminating the electromagnetic energy exposure after detecting the visual and/or textural changes consistent with sterilization or disinfection of the article adjacent the indicator element.

An operator may detect the change in the indicator element and manually turn off or otherwise discontinue the exposure, or optionally a sensor may be used to detect the change in the indicator element and provide a signal to a control system that automatically terminates the exposure. This may be a mechanical control system, an electromechanical control system, or an electrical system.

The indicator element may comprise a photochromic surface or material, and the visual and/or textural changes may be reversible or irreversible.

The electromagnetic energy may comprise light with a wavelength between about 100 nm to about 120 nm, about 120 nm to about 140 nm, about 140 nm to about 160 nm, about 160 nm to about 180 nm, about 180 nm to about 200 nm, about 200 nm to about 220 nm, about 220 nm to about 240 nm, about 240 nm to about 260 nm, or about 260 nm to about 280 nm. The electromagnetic energy may comprise light with a wavelength between about 240 nm to about 260 nm, or with a wavelength that is about 254 nm.

The electromagnetic energy may be provided from a source providing an output of about 100 watts to about 1000 watts, or an output of about 800 watts. The article being sterilized or disinfected and/or the indicator element may be exposed to a dose of electromagnetic energy between about 10,000 to about 60,000 μW-sec/cm². The article being sterilized or disinfected and/or the indicator element may be exposed to electromagnetic energy for between about 0.01 seconds to about 120 seconds, or for between about 10 seconds to about 90 seconds. The article being sterilized or disinfected and/or indicator element may be positioned within about 0.5 to about 12 feet from a source of the electromagnetic energy.

The indicator element may comprise an adhesive strip, a pod structure, a panel, tubing, woven fabric, nonwoven fabric, paper, wrapper, or a surface coating that may be painted or otherwise applied to the article being sterilized or disinfected. Optionally, the indicator element may be integral with the article so that there is a single assembly. The article being sterilized or disinfected may be selected from a piece of furniture, a computer, a wall, a counter, a piece of diagnostic equipment, a piece of medical equipment, a piece of laboratory equipment, a railing, a sink, a toilet, a shower, a trash receptacle, a surgical instrument, a telephone, a remote control, a light switch, bedding, and an electrocardiograph. The article being sterilized or disinfected may be selected from a door handle, a hospital bed, a patient bed, an operating table, a medical tray, a med cart, a wound cart, a wheelchair, a food tray, a perfusion pump, endoscopy equipment, ventilator equipment, a heart-lung bypass machine, a medical light, a medical vacuum system, a surgical lamp, an operating room light, an operating table, an oxygenizer, anesthetic equipment, decontamination equipment, an examination chair, an examination lamp, an examination table, an incubator, an autoclave, a heart-lung machine, a patient monitor, a patient supervision system, a transport stretcher, an ultrasonic atomizer, an X-ray apparatus, and an electrocardiograph. The piece of furniture may be a bed, a table, or a chair.

The exposing may occur in a medical care facility, and exposure to the electromagnetic energy may result in sterilization or disinfection of at least one surface of the article. The sterilization or disinfection also may comprise at least one of air disinfection, water disinfection, or surface disinfection. The sterilization or disinfection may be characterized by a microbial kill efficacy of at least 85%, at least 88%, at least 90%, at least 91%, at least 92%, at least 93%, at least 94%, at least 95%, at least 96%, at least 97%, at least 98%, at least 99%, at least 99.2%, at least 99.5%, at least 99.9% or at least 99.9999%. Sterilization or disinfection may result in inactivation of one or more bacteria, molds, protozoa, fungi, viruses, or yeasts, or any combination thereof.

The indicator element may be a photochromic surface that may comprise a photochrome that is a photochromic monomer, a photochromic oligomer, or a photochromic polymer where the polymer contains a photochromic material either chemically bonded to, or blended with the polymer. The photochromic surface may comprise a photochrome that is a photochromic small molecule having a molecular weight of less than 1000 Daltons. The small molecule may be a pyran or a spiropyran such as benzo[3,4]fluoreno[2,1-b]pyran-13-ol, 3,13-dihydro-3,3-bis(4-methoxyphenyl)-6,11,13-trimethyl-,

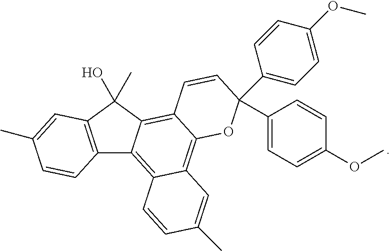

The photochrome may comprise an optionally substituted coumarin moiety, an optionally substituted spirooxazine moiety, an optionally substituted naphthoaxazine moiety, an optionally substituted naphthopyran moiety, an optionally substituted phenoxyanthraquinone moiety, an optionally substituted carbazole moiety, or an optionally substituted spiropyran moiety, or combinations thereof.

The indicator element may be releasably coupled to the article being sterilized or disinfected, or it may be fixedly coupled thereto. The method may also further comprise providing a blocking agent and preventing exposure of the indicating element or a photochromic layer within the indicating element from ultraviolet A and ultraviolet B energy.

An indicator element for use in any of the methods described herein may comprise a photochromic surface.

In another aspect of the present invention, an indicator for indicating sterilization or disinfection of an article comprises an indicator element configured to react to and indicate exposure to electromagnetic energy, wherein the electromagnetic energy has a wavelength between about 100 nm and about 280 nm, and wherein the exposure results in one or more visual and/or textural changes to the indicator element. The indicator also comprises a separate layer or substrate coupled to the indicator element and wherein the substrate is configured to be coupled to an article being sterilized or disinfected.

In still another aspect of the present invention, a system for indicating sterilization or disinfection of an article comprises an indicator element configured to react to and indicate exposure to electromagnetic energy, wherein the electromagnetic energy has a wavelength between about 100 nm and about 280 nm, and wherein the exposure results in one or more visual and/or textural changes to the indicator element. The system also comprises a source of the energy, such as electromagnetic energy for sterilizing or disinfecting the article.

The indicator element may comprise a photochromic surface or material. The photochromic surface may comprise a photochrome that is a photochromic monomer, a photochromic oligomer, or a photochromic polymer wherein the polymer includes a photochromic molecule blended with or chemically boned to the polymer. The photochromic surface may comprise a photochrome that is a photochromic small molecule having a molecular weight of less than 1000 Daltons.

The photochromic small molecule may be a pyran or a spiropyran such as benzo[3,4]fluoreno[2,1-b]pyran-13-ol, 3,13-dihydro-3,3-bis(4-methoxyphenyl)-6,11,13-trimethyl-,

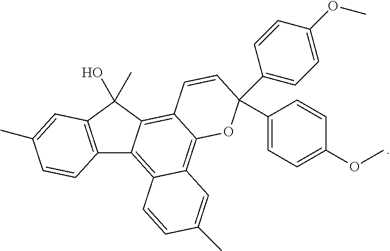

The photochrome may comprise an optionally substituted coumarin moiety, an optionally substituted spirooxazine moiety, an optionally substituted naphthoaxazine moiety, an optionally substituted naphthopyran moiety, an optionally substituted phenoxyanthraquinone moiety, an optionally substituted carbazole moiety, or an optionally substituted spiropyran moiety, or combinations thereof.

The indicator element may be configured as one or more layers of material, and the layers of materials may comprise a polymer substrate layer. The substrate layer may be a discrete layer from the indicator element. The substrate layer may comprise an adhesive for coupling the indicator to the article being sterilized or disinfected.

The visual and/or textural changes may be irreversible or they may be reversible. The indicator element may comprise an adhesive strip, a pod structure, a panel, tubing, woven fabric, nonwoven fabric, paper, wrapper, or a surface coating. The visual and/or textural changes may indicate sterilization or disinfection of the article characterized by a microbial kill efficacy of at least 85%, at least 88%, at least 90%, at least 91%, at least 92%, at least 93%, at least 94%, at least 95%, at least 96%, at least 97%, at least 98%, at least 99%, at least 99.2%, at least 99.5%, at least 99.9% or at least 99.9999%.

The indicator may further comprise a blocking agent for blocking ultraviolet A and ultraviolet B energy from a photochromic layer within the indicator element.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a top view of an article comprising a photochromic surface.

FIG. 1B shows a side view of the article in FIG. 1A.

FIG. 1C shows a magnified side view of section 18 in FIG. 1B

FIG. 2A shows a side view of an exemplary article having a photochromic surface prior to exposure to UV-C energy.

FIG. 2B shows the article of FIG. 2A during exposure to UV-C energy.

FIG. 3A depicts a color change that occurs on an indicator element such as a photochromic surface, after varying degrees of exposure to UV-C energy.

FIG. 3B shows an exemplary article pre-exposure to UV-C energy.

FIG. 3C shows the exemplary article of FIG. 3B after exposure to UV-C energy.

FIG. 4 illustrates an experimental setup for measuring UV transmission through various polymer films.

FIG. 5 illustrates the relationship between UV-C transmission and film thickness.

FIGS. 6A-6D illustrate optional configurations of an indicator element.

### DETAILED DESCRIPTION

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Approximately and About: As used herein, the term “approximately” and the term “about” are intended to encompass normal statistical variation as would be understood by those of ordinary skill in the art. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Photochrome: As used herein, the term “photochrome” refers to any chemical species that may transform between two forms as a result of exposure to a particular form of electromagnetic energy. For example, optionally, a photochrome is a molecule that transforms between two forms in response to exposure to UV light. Optionally, the transformation of a photochrome results in a color change, for example, a gain or loss of color or change in the perceived color of the photochrome, or a substance comprising the photochrome. Optionally, a photochrome will exhibit one or more of the following properties: the ability to change absorption spectra and/or refractive index, modulation of molecular fluorescence yield, and/or optically induced structural changes in supramolecular complexes (see Barkauskas et al., Ultrafast dynamics of photochromic compound based on oxazine ring opening, Lithuanian J Physics, 48(3): 231-242 (2008) for a discussion of certain photochromic molecules). Optionally, the transformation of a photochrome is reversible, for example, via thermal back conversion or exposure to light of a certain wavelength or wavelengths. Unless otherwise specified, the terms “photochrome” and “photochromic molecule” are used interchangeably.

Small molecule: As used herein, the term “small molecule” means a low molecular weight organic compound that may serve as a photochromic molecule or portion thereof. In general, a “small molecule” is a molecule that is less than about 5 kilodaltons (kD) in size. Optionally, provided nanoparticles further include one or more small molecules. Optionally, the small molecule is less than about 4 kD, 3 kD, about 2 kD, or about 1 kD. Optionally, the small molecule is less than about 800 daltons (D), about 600 D, about 500 D, about 400 D, about 300 D, about 200 D, or about 100 D. Optionally, a small molecule is less than about 2000 g/mol, less than about 1500 g/mol, less than about 1000 g/mol, less than about 800 g/mol, or less than about 500 g/mol. Optionally, one or more small molecules are incorporated into, or otherwise associated with, a nanoparticle, polymer matrix, or other matrix. Optionally, small molecules are non-polymeric, or optionally the small molecules may be chemically bonded or blended with a polymer.

Substantially: As used herein, the term “substantially” refers to the qualitative condition of exhibiting total or near-total extent or degree of a characteristic or property of interest. One of ordinary skill in the relevant art will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Described herein, among other things, are exemplary methods for photochromic detection of electromagnetic energy (e.g., ultraviolet radiation) using photochromic surfaces, as well as exemplary indicator elements suitable for use in such methods. Optionally, detection of exposure to electromagnetic energy, such as UV-C radiation, may be used to determine and/or infer the sterilization or disinfection status of an article, area, or surface. It is well-known in the art that certain forms of electromagnetic energy, such as UV light and specifically UV-C, has potent germicidal properties.

Optionally, the present disclosure provides methods for sterilizing or disinfecting an article, including:

(a) providing an indicator element adjacent the article comprising a photochromic surface or material;

(b) exposing said article and the indicator element to electromagnetic energy having a wavelength preferably between about 100 nm and about 280 nm, more preferably between about 200 nm-280 nm, or even more preferably between about 240 nm-270 nm, where the exposure results in one or more visual and/or textural changes to said indicator element;

(c) detecting the one or more visual and/or textural changes of said indicator element that optionally comprises one or more photochromic surface or one or more materials; and

(d) terminating the ultraviolet radiation exposure after the visual and/or textural changes are consistent with sterilization or disinfection of the article and/or indicator element in correlation with dosage of ultraviolet radiation.

Optionally, the present disclosure also provides indicator elements for use in any of the methods described herein, wherein said indicator element may comprise a photochromic surface or material.

Sterilization or Disinfection by Exposure to Electromagnetic Energy

Exposure to electromagnetic energy such as ultraviolet light/radiation is a known method by which to sterilize or disinfect certain environments by deactivating/killing environmental pathogens (e.g., pathogens present on a surface or pathogens which are airborne). The wavelength of ultraviolet radiation ranges from 100 nm to 280 nm, with UV light having wavelengths between about 240 nm and about 260 nm being particularly useful to achieve the desired germicidal effect. Ultraviolet germicidal irradiation (UVGI) methods can be particularly useful in, for example, medical, residential, commercial, or manufacturing facilities. Without wishing to be held to a particular theory, it is thought that the germicidal effects result, at least in part, from the disruption of the DNA of microbial pathogens (e.g., bacteria, bacterial spores, and viruses, including those described herein). Specifically, it is thought that exposure to UVGI alters pyrimidine bases, such as cytosine and thymine, which have conjugated double bonds and as such absorb UV light, which results in one of two products. The first product is the formation of a cyclobutane ring between two pyrimidines. For example, formation of cysteine cyclobutane photodimers may result in formation of thymine dimers via the SOS response system in both prokaryotic and eukaryotic organisms. The second product is formation of a (6-4) pyrimidine. The formation of one or both of these products within the structure of DNA in turn is thought to result in inhibition of proper transcriptional and replicational templates through the destruction of the molecular bonds of the nucleic acids therein and resulting in the formation of thymine dimers. More information regarding the effects of UVGI on prokaryotic and/or eukaryotic DNA may be found, inter alia, in Fu et al., 1997, Applied and Environ Microbiol, 63(4): 1551-1556; Fu et al., 2008, FEMS Microbial Rev 32(6): 908-926; and Eller and Gilchrest, 2000, Pigment Cell Res, 13 Suppl 8: 94-97.

Electromagnetic energy can be provided by various sources, such as portable ultraviolet lamps. Exemplary sources of UV-C radiation include those described in International Application No. PCT/US2013/076717, filed Dec. 19, 2013, now published as WO 2014/100493.

Optionally, exposure to electromagnetic energy results in one or more of, air ozonation, air oxygenation, chlorine removal, or carbon reduction. Such effects are known to be beneficial, inter alia, in medical or other healthcare facilities.

Sterilization or disinfection may be characterized or defined in different ways. Optionally, sterilization or disinfection comprises air disinfection, water disinfection, or surface disinfection. In other embodiments, the sterilization or disinfection is characterized by a microbial kill efficacy of at least 85%, at least 88%, at least 90%, at least 91%, at least 92%, at least 93%, at least 94%, at least 95%, at least 96%, at least 97%, at least 98%, at least 99%, at least 99.2%, at least 99.5%, or at least 99.9%, or at least 99.9999%.

Optionally, sterilization or disinfection may be characterized or defined as the inactivation of at least one pathogen in or on an article, area, or surface (e.g., the sterilization or disinfection results from the inactivation of one or more bacteria, molds, protozoa, viruses, or yeasts, or any combination thereof). It is contemplated that any bacteria, mold, protozoa, fungi, virus, yeast, or combination thereof may be inactivated via the use of UVGI. Optionally, the pathogen is norovirus, hepatitis B virus, Acinetobacter spp, Pseudomonas aeruginosa, Clostridium difficile, and/or Candida spp. In certain embodiments, the pathogen is vegetative bacteria (e.g., methicillin-resistant Staphylococcus aureus (MRSA), vancomycin-resistant Enterococcus (VRE), or Acinetobacter such as Acinetobacter baumannii). Optionally, the sterilization or disinfection results from the inactivation of one or more of methicillin-resistant Staphylococcus aureus (MRSA), vancomycin-resistant Enterococcus (VRE), Acinetobacter spp., Clostridium difficile (C. difficile), carbapenemase-resistant Klebsiella pneumonia (KPC), multi-drug resistant Pseudomonas aeruginosa, Acinetobacter baumannii, C. albicans, C. glabrata, C. parapsilosis, C. krusei, Aspergillus fumigatus, Fusarium solani, Scedosporium apiospermum, or norovirus.

In other embodiments, sterilization or disinfection may be characterized or defined as the inactivation of one or more multidrug-resistant organisms (MDRO). Optionally, the MDROmay be Staphylococcus aureus (MRSA) or Pseudomonas aeruginosa.

Photochromism and Exemplary Photochromes

Optionally, the invention provides indicator elements that comprise a photochromic structure (e.g., a photochromic surface) that responds to one or more forms of electromagnetic energy, and uses of such indicator elements. For example, a photochromic surface or material may react in the presence of electromagnetic energy that falls in the ultraviolet-C range (e.g., between about 100 nm and about 280 nm, more preferably between about 200 nm-280 nm, or even more preferably between about 240 nm-270 nm). According to various options, such photochromic surfaces then produce one or more useful feedback mechanisms to indicate the success and/or degree of a sterilization or disinfection process that includes UV-C radiation. Optionally, a feedback mechanism may include a visual change, for example, a color change. Optionally, a feedback mechanism may include a textural change, for example, a roughening or smoothing of a surface. Optionally, the feedback mechanism(s) may vary according to one or more parameters that affect the dose and/or intensity of UV-C including but not limited to the UV intensity, proximity of the UV radiation source, and duration of exposure. The feedback mechanism is not limited to a photochromic material, and may optionally be any indicator element which provides visual, audio, tactile, odor, taste, or other feedback to an operator that the desired level of exposure to the electromagnetic energy such as UV-C has been achieved, or that a desired level of sterilization or disinfection has been achieved.

Optionally, a sensor may also be used to detect the change in the indicator element and the sensor may then send a signal over a wire, or wirelessly to a controller that can control and allow continued exposure, or that can discontinue the exposure. For example, optionally a photodetector may be used to detect color change in the indicator element and this initiates a feedback process that may automatically terminate the UV exposure when a desired exposure level is detected, or allows continued exposure until the desired exposure level is reached and/or detected.

Optionally, a photochromic structure (comprising at least one photochrome) may be activated when exposed to UV-C radiation that includes wavelengths of about 100 nm-280 nm, more preferably between about 200 nm-280 nm, or even more preferably between about 240 nm-270 nm. These ranges may vary by plus or minus 20%. In certain options, the UV-C radiation includes a wavelength of about 254 nm, which is the wavelength known to have the highest germicidal activity. Without wishing to be held to a particular theory, such ranges are thought to correspond to the germicidal region of UV radiation and can inactivate many pathogens. The extent of pathogen inactivation can be optimized through the variation of various parameters, including those mentioned above such as UV intensity, proximity and duration of exposure from a single source or an aggregate of sources with radial or multivector emissions. Such targeted activation can therefore provide a completed sterilization or disinfection feedback mechanism, where the photochromic response or other indicator element response can provide feedback to an operator (e.g., visual and/or textural feedback) when the desired combination of parameters such as UV intensity, proximity and duration of exposure has been achieved (e.g., a sterilization or disinfection process has been accomplished at the desired levels of efficacy).

For example, activation and/or alteration of a photochrome may begin just after initiation of exposure to the electromagnetic energy (e.g., UV-C radiation). During the exposure from time t=0 seconds until the appropriate level and combination of UV-C light intensity and exposure has been reached, the photochrome or other indicator element may become more prominently activated as the levels of intensity and exposure reach a desired state over the duration of exposure. The photochromic response (e.g., a textural or visual change such as a color change) may then indicate when the desired effect of exposure to UV-C has been accomplished. Accordingly, the operator will recognize that the desired level of sterilization or disinfection has been completed via the excitation (e.g. color change) of the indicator element which comprises a photochrome.

Optionally, a photochromic structure may be designed or formed such that a photochromic response (e.g., a visual or textural activation) may occur only a single time, or for a single use, meaning that the photochromic structure does not revert to its normal or inactivated state and the activation is therefore irreversible.

Optionally, a photochromic structure may be designed or formed such that a photochromic response (e.g., a visual or textural activation, or other response) may occur several times (e.g., for repeated cycles); that is, the photochromic response is reversible. For example, the visual or textural activation may dissipate over time, returning the photochromic structure to the textural or visual state it was in prior to exposure to UV-C, once the sterilization or disinfection or UV-C exposure has been completed. Therefore, the indicator may be biased to return to its unexposed condition to allow reuse.

An indicator element such as a photochromic surface may be applied to, or incorporated in any suitable article. According to various embodiments, a photochromic surface may be applied to an article or surface to be sterilized or disinfected and it may be in various forms, including but not limited to an adhesive strip, a pod structure, a card, a panel, tubing, woven fabric, nonwoven fabric, paper, wrapper, or a surface coating.

Optional Configurations

An adhesive strip: To be placed on articles in one or multiple locations. The adhesive strip would have a top portion containing the indictor element and the bottom portion would contain an adhesive. The adhesive could be a strong long lasting substance for a more permanent fixation to the article or surface being exposed to the UV-C energy. For this application, the indicator element can be reversible or irreversible. The adhesive could be temporary for a one time use and then the adhesive strip can be easily removed from the surface being sterilized or disinfected. In the case where the adhesive strip is removed leaving an unsterilized or non-disinfected surface, that surface may be sterilized or disinfected by further exposure to UV-C energy or by other methods known in the art and in accordance with standard operating procedures, if desired.

Pod Structure or Badge: The indicator may be any size, but preferably is large enough to allow easy reading from a reasonable distance. For example, the indicator may be about 3 inches long x about 2 inches wide x about 0.25 inches thick and with the top surface of the pod, badge or card containing the indicator element. The pod, card or badge can be worn or placed on an article or surface being exposed to the UV-C energy. The pod, card or badge can have a strong or temporary adhesive to the side in contact with the article that is receiving UV-C energy with the side containing the indicator element facing the UV-C source. Optionally, the pod, card, or badge may have a thin profile (e.g. like a credit card or thinner), and may be a flat planar square, rectangular, oval, round, circular, polygonal, triangular, spherical or any other desired shape.

Wrapper: for this option, the indicator element would be spread throughout the entire top surface or in one or more discrete areas of the wrapper or sheet. The wrapper or sheet would be used to cover an object in a manufacturing or healthcare setting where the object is receiving UV-C energy. The wrapper or sheet can be of different lengths and widths depending on the size of the article being wrapped. The thickness could range from thin to thick depending on the application requirements. Thinner sheets add less bulk during wrapping and are easier to wrap around an article, but the thicker wrappers are less likely to be punctured or torn during use.

Surface Coating: Optionally, the indicator elements are dispersed in a liquid and may be applied as a surface coating to an article that receives exposure to UV-C energy. The indicator element could be painted or sprayed onto a surface in a single layer or with a process of several layers.

Fabric: Optionally, the fabric is constructed with strands of indicator element with uninform or varying diameters depending on the application. Diameter and concentration properties would be adjusted for adjusting to strength and overall thickness properties desired with the combination of individual indicator element strands. The strands can be woven or non-woven to make a fabric like mesh. The strands can be combined to formulate sheaths of fabric for wearable applications, or in cellular or biological applications including implantable grafts, surgical mesh, surgical tubing, or other medical devices and implants or prostheses. Optionally, the indicator element is a discrete element that is attached to the fabric such as by stitching, adhesives, etc.

Optionally, selection of an indicator element such as a photochromic molecule for a particular purpose or purposes may be based on one or more of the following factors: exposure source/wavelength of light to which the molecule reacts; the intensity of visual (e.g., color) change exhibited by the photochromic molecule in response to UV-C; the time it takes for a photochromic molecule to display a visual change at a desired intensity and/or for a desired duration; the number of activation cycles a photochromic molecule can sustain before the reaction of the photochromic molecule to UV-C substantially degrades; and the specific visual and/or textural change exhibited by a photochromic molecule. Optionally, selection of a desired photochromic molecule for use will include consideration of any of the above factors. Optionally, two or more of the factors above may be considered in selecting a photochromic material.

While any of a variety of photochromes may be used in accordance with any options, several exemplary photochromic molecules are recited herein to more clearly identify the scope of certain options. Generally, a photochromic molecule selected for use optionally, may be, inter alia, a spiropyran, spirooxazine, chromene, fulgide/fulgimide, diarylethene, azo compound, spirodihydroindolzine, polycyclic aromatic compound, anil, polycyclic quinone, triarylmethane, viologen, or perimidinespirocyclohexadienone. Optionally, a photochromic molecule may be selected from: 3,3-dimethyl-1-methyl-2,2′-[2H]bipyrido-[3,2-f][2,3-h][1,4]benzoxazine; 2,3,3-trimethyl-3H-indolium iodide; 1′,3′,3′-trimethyl-6-nitrospiro[chromene-2,2′-indolene]; 1,2-bis(2,4-dimethyl-5-phenylthiopen-3-yl)perfluorocyclopentene; N,N-dimethyl-2-[6-(4-nitrophenyl)-2-phenyl-1,3-diazabicyclo[3.1.0]hex-3-en-2-yl]benzenamine; MB131 Spirophyran. Optionally, a photochromic molecule may be a form of Reversacol such as Reversacol Claret (Product Code 993-601-50; Keystone), Reversacol Berry Red (Product Code 993-600-50; Keystone), Reversacol Cinnibar (Product Code 993-600-52; Keystone), Reversacol Flame (Product Code 993-604-50; Keystone). Optionally, a photochromic molecule may be ADA7226 (HW Sand Corp.).

Some optional photochromes suitable for the methods and articles described herein are included in the following Table 1, and/or in the options described in the references cited therein in order to better illustrate the principles of some embodiments of the invention. Other photochromes suitable for use in, among other applications, medically relevant options are described in, e.g., Meyering et al., U.S. Patent Application Publication No. 2012/0082713. Optionally, a photochromic surface comprises polyethylene or a polyethylene analog or derivative.

TABLE 1
Exemplary Photochromes

| Photochromic | | |
|---|---|---|
| Substructure | Exemplary Chemical Structure | Reference |
| coumarin | 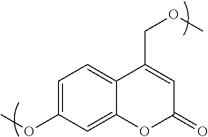 | Maddipatla et al., Macromolecules, 2013, 46 (13), pp 5133-5140. |
| coumarin | 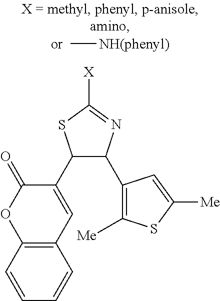 | Traven et al., Organic Letters, 2008, 10, 1319-1322. Meyering et al., U.S. Patent Application Publication No. 2012/0082713 |
| coumarin/spiropyran | 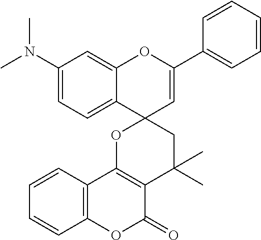 [SMILES: CN(C)c1ccc2c(c1)OC(c1ccccc1)=CC21CC(C)(C)c2c(c3ccccc3oc2=O)O1] | Chen et al., Organic Letters, 2008, 10(21), 4823-2826. |
| coumarin co- polymers | 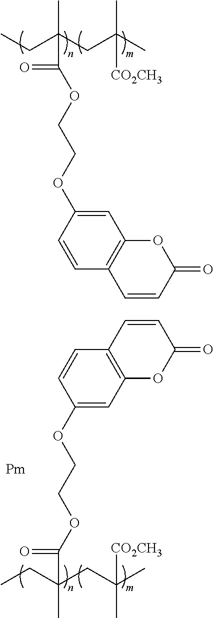 | Iliopoulous et al., J. Am. Chem. Soc., 2010, 132, 14343-14345. |
| spirooxazine | 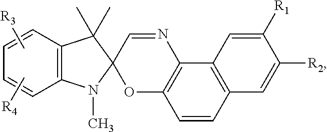 | Chu et al., U.S. Pat. No. 4,215,010 |
| | where one of R₁ and R₂ is halogen | |
| | or low alkoxy, and the other is | |
| | hydrogen, and R₃ and R₄ are | |
| | hydrogen, lower alkyl, lower | |
| | alkoxy, or halogen. | |
| spirooxazine | 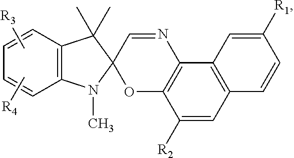 | Chu et al., U.S. Pat. No. 4,699,473 |
| | where one of R₁ and R₂ is a lower | |
| | alkoxy group and the other is | |
| | hydrogen; R₃ is a trifluoromethyl | |
| | group; and R₄ is hydrogen, a lower | |
| | alkyl or a lower alkoxy group. R₃ | |
| | and R₄ are independently located at | |
| | the 4, 5, 6, or 7 position of the | |
| | indoline ring | |
| spirooxazine | 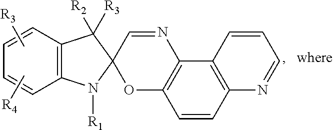 | Hurditch et al., U.S. Pat. No. 4,637,698 |
| | (a) R₁ is selected from the group | |
| | consisting of C₁-C₈ alkyl, phenyl, | |
| | phen(C₁-C₄) alkyl, allyl and | |
| | mono- and di-substituted phenyl, | |
| | said substituents being selected | |
| | from C₁-C₄ alkyl and C₁-C₅ | |
| | alkoxy; | |
| | (b) R₂ and R₃ are each selected | |
| | from the group consisting of C₁- | |
| | C₅ alkyl, phenyl, C₁-C₄ alkyl and | |
| | C₁-C₅ alkoxy mono- and | |
| | disubstituted phenyl, benzyl or | |
| | combined to form a cyclic ring | |
| | selected from the group consisting | |
| | of an alicyclic ring containing from | |
| | 6 to 8 carbon atoms (including the | |
| | spiro carbon atom), norbornyl and | |
| | adamantyl, and | |
| | (c) R₄ and R₅ are each selected | |
| | from the group consisting of | |
| | hydrogen, C₁-C₅ alkyl, halogen, | |
| | C₁-C₅ alkoxy, nitro, cyano and C₁- | |
| | C₈ alkoxycarbonyl | |
| naphthoaxazine | 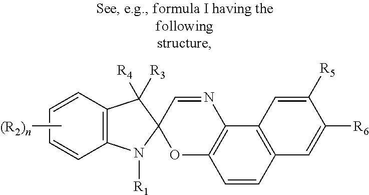 | Van Gemert, WO 1996/011926 |
| naphthoaxazine | 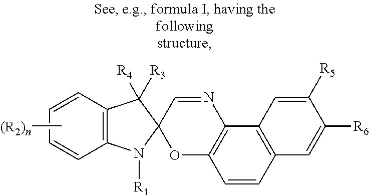 | Van Gemert, U.S. Pat. No. 5,405,958 |
| naphthopyran | 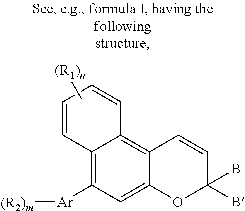 | Lin et al., WO 1999/031082 |
| naphthopyran | 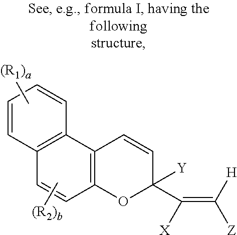 | Van Gemert, WO1994/007889 |
| naphthopyran | 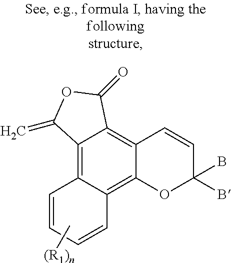 | Lin, WO 1999020629 |
| naphthopyran | 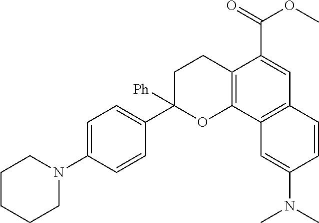 [SMILES: COC(=O)c1cc2ccc(N(C)C)cc2c2c1CCC(c1ccccc1)(c1ccc(N3CCCCC3)cc1)O2] | Nunzio et al., Chem. Phys. Chem. 9, 768- 775, 2008. |
| naphthopyran | See above | Delbaere et al., Org. Lett., 8, 4931- |
| | | 4934, |
| | | 2006. |
| Phenoxyanthraquinone | N/A | Liu et al., Dyes and Pigments, 35(3), |
| | | 279- |
| | | 288, 1997. |
| carbazole | 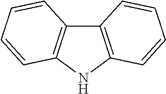 [SMILES: c1ccc2c(c1)[nH]c1ccccc12] | Altomare et al., Macromolecular Chemistry and Physics, 205(12), 1611- 1619. |
| spiropyran | 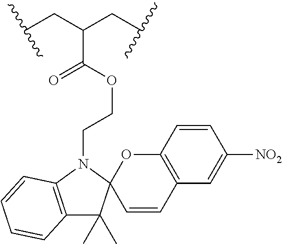 [SMILES: *CC(C*)C(=O)OCCN1c2ccccc2C(C)(C)C12C=Cc1cc([N+](=O)[O-])ccc1O2] | Zhu et al., J. Am. Chem. Soc. 128(13): 4303-4309, 2006. |
| spiropyran | 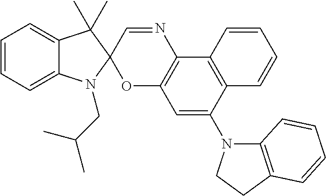 [SMILES: CC(C)CN1c2ccccc2C(C)(C)C12C=Nc1c(cc(N3CCc4ccccc43)c3ccccc13)O2] | Nunzio et al., Chem. Phys. Chem. 9, 768- 775, 2008. |
| spiroperimidine | 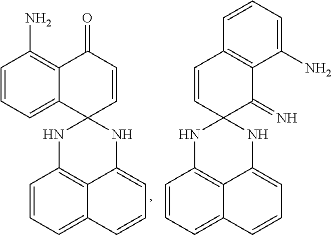 | Meyering et al., U.S. Patent Application Publication No. 2012/0082713 |
| diarylethene | 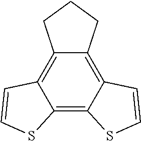 [SMILES: c1cc2c3c(c4ccsc4c2s1)CCC3] | Meyering et al., U.S. Patent Application Publication No. 2012/0082713 Cipolloni et al, J. Phys. Chem. 112, 4764- 4771, 2008. |

Optionally, a photochromic surface or material comprises a photochrome that is a photochromic monomer, a photochromic oligomer, or a photochromic polymer.

Optionally, a photochromic surface or material comprises a photochrome that is a photochromic small molecule having a molecular weight of less than 5 kDa. Optionally, a photochromic small molecule has a molecular weight of less than 4 kDa, 3 kDa, 2 kDa, or 1 kDa.

Optionally, a photochrome comprises an optionally substituted coumarin moiety, an optionally substituted spirooxazine moiety, an optionally substituted naphthoaxazine moiety, an optionally substituted naphthopyran moiety, an optionally substituted phenoxyanthraquinone moiety, an optionally substituted carbazole moiety, or an optionally substituted spiropyran moiety, an optionally benzofluorenylpyranol moiety, or combinations thereof.

Optionally, a photochromic small molecule is such as a pyran or spiropyran such as benzo[3,4]fluoreno[2,1-b]pyran-13-ol, 3,13-dihydro-3,3-bis(4-methoxyphenyl)-6,11,13-trimethyl-,

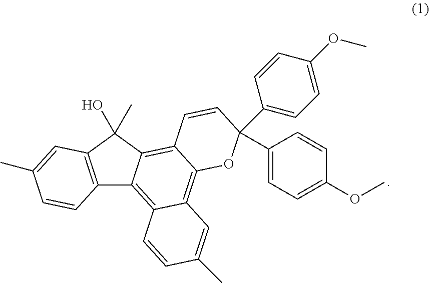

Optionally, Compound (1) can be modified by variation of the methyl and/or methoxy substituents (e.g., replacement of 1, 2, 3, or 4 of these groups with a group selected from CN or other compounds known in the art.

Optionally, a photochromic surface or material is releasably coupled to the article. In still other embodiments, a photochromic surface or material is fixedly coupled to the article.

Electromagnetic Energy

The use of electromagnetic energy, such as UV-C light, to sterilize or disinfect an article, area, or surface is known to depend upon several factors including the intensity of the electromagnetic energy, the length of time the article, area, or surface is exposed to the energy, and the proximity of the article, area, or surface to the source of the electromagnetic energy. Various options will include variations of one or more of these parameters in order to achieve a proper dose for a particular application. Optionally, a particular application may include partial or complete sterilization or disinfection of an article, area or surface.

According to various options, electromagnetic energy may be of any of several forms. Optionally, electromagnetic energy is light of a particular wavelength or range of wavelengths. Optionally, the electromagnetic energy comprises light with a wavelength between about 100 nm to about 120 nm, about 120 nm to about 140 nm, about 140 nm to about 160 nm, about 160 nm to about 180 nm, about 180 nm to about 200 nm, about 200 nm to about 220 nm, about 220 nm to about 240 nm, about 240 nm to about 260 nm, or about 260 nm to about 280 nm. In certain options, the electromagnetic energy comprises light with a wavelength between about 200 nm to about 270 nm. In other options, the electromagnetic energy comprises light with a wavelength between about 200 nm to about 320 nm. In other options, the electromagnetic energy comprises light with a wavelength between about 240 nm to about 260 nm. Optionally, the electromagnetic energy comprises light with a wavelength of approximately 254 nm.

Various options will use one or more sources of electromagnetic energy. A variety of sources of electromagnetic energy (e.g., sources of UV-C light) are contemplated as within the scope of the present disclosure. Optionally, the source of electromagnetic energy has an output of 0.5-2,500 watts (W). Optionally, the source of electromagnetic energy has an output of between 100 W and 1,000 W. Optionally, the source(s) of electromagnetic energy has an output of about 500 W to about 2,500 W (e.g., about 500 W, about 600 W, about 700 W, about 800 W, about 900 W, or about 1000 W. In certain options, the source of electromagnetic energy has an output of about 800 W. Optionally, the source of electromagnetic energy has an output of between 10 W and 100 W (e.g., between 10 W and 90 W, between 10 W and 80 W, between 10 W and 70 W, between 10 W and 60 W, between 10 W and 50 W, or between 10 W and 40 W). Optionally, the source of electromagnetic energy has an output between 400 and 2,500 W.

The dose of electromagnetic energy administered will vary according to the options used in a particular application. For example, the dose of electromagnetic energy used will vary if partial sterilization or partial disinfection is desired as opposed to substantially complete sterilization or disinfection. Further, the dose of electromagnetic energy used may vary if only one or a few types of microbes are being targeted for destruction. Optionally, the electromagnetic energy is provided at a dose of about 500 to about 450,000 μW-sec/cm². In certain options, the dose is about 500 to about 5,000 μW-sec/cm², e.g., about 500 to about 2,500 μW-sec/cm², or about 1,000 to about 2,000 μW-sec/cm². Optionally, the electromagnetic energy is provided at a dose or between about 10,000 to about 30,000 μW-sec/cm², e.g., from about 15,000 to about 30,000 μW-sec/cm², from about 20,000 to about 30,000 μW-sec/cm², or from about 20,000 to about 26,000 μW-sec/cm². Optionally, the electromagnetic energy is provided at a dose of at least 5,000 μW-sec/cm².

Various options will include exposure to electromagnetic energy for various amounts of time, according to the needs of a particular application. Optionally, the article, area, or surface is exposed to electromagnetic energy for between about 0.01 seconds to about 120 minutes. Optionally, the article, area, or surface is exposed to electromagnetic energy for between about 0.01 seconds to 60 minutes. Optionally, the article, area, or surface is exposed to electromagnetic energy for between about 1 second and 15 minutes e.g., from about 1 second to about 10 minutes, about 1 second to about 5 minutes, or about 5 seconds to about 5 minutes. Optionally, the article, area, or surface is exposed to electromagnetic energy from about 1 second to about 120 seconds, e.g., from about 1 second to about 90 seconds, from about 1 second to about 60 seconds, from about 5 seconds to about 60 seconds, or from about 10 seconds to about 60 seconds.

It is contemplated that the distance between a source of electromagnetic energy (e.g., UV-C light) and an article, area, or surface to be sterilized or disinfected will vary according to the requirements of a particular application. Optionally, the article, area, or surface to be sterilized or disinfected is positioned within about 0.5 to about 15 feet from the source of the electromagnetic energy, e.g., from within about 0.5 to about 10 feet from the source of electromagnetic energy. Optionally, the article, area, or surface to be sterilized or disinfected is positioned from within about 0.5, 1, 2, 3, 4, 5, 6, 7, 8, 9, 10, 11, or 12 feet from the source of the electromagnetic energy.

### Exemplary Articles to be Sterilized or Disinfected

Any of a wide variety of articles, areas, and/or surfaces may be sterilized or disinfected. It is contemplated that any article, area or surface that would benefit from sterilization or disinfection may be used according to various options. Optionally, an article to be sterilized or disinfected is selected from: a piece of furniture, a computer, a wall, a counter, a piece of diagnostic equipment, a piece of medical equipment, a piece of laboratory equipment, a railing, a sink, a toilet, a shower, a trash receptacle, a surgical instrument, a telephone, a remote control, a light switch, and bedding. Optionally, a piece of furniture may comprise a bed, a bedside rail, a table (e.g., an operating or examination table), or a chair (e.g., a wheelchair or examination chair). Optionally, the article to be sterilized or disinfected is the interior of a drawer, cabinet, shelf, closet, or other storage unit. Optionally, the article to be sterilized or disinfected is selected from: door handles, medical trays, medical carts, wound or supply carts, food trays, a sink, a toilet, a shower, or bathroom (e.g., a bathroom floor), perfusion pumps, endoscopy equipment, ventilator equipment, heart-lung bypass machine, or electrocardiograph (ECG).

Examples of commercial applications include but are not limited to those described below.

Food processing and Manufacturing: The indicator element can be added to the wrapping materials used for packaging and storing food as the food passes through UV-C energy conveyer belt systems.

Pharmaceutical Manufacturing: The indicator strips or pods can be placed in laboratory and manufacturing facilities on articles and surfaces that are consistently exposed to UV-C energy for controlling the exposure of products to bacteria or foreign organisms during the manufacturing process. The indicator strips or pods would be an excellent way to demonstrate disinfection process throughout the facilities.

Aeronautics and Outer Space Exploration: Astronauts, gear, equipment, and materials brought to outer space receive varying levels of UV-C energy. Indicator elements in pod, fabric, or strip configurations can be utilized to measure the exposure to UV-C energy during the time in outer space.

In certain options, an article to be sterilized or disinfected may be: a medical light, a medical vacuum system, a surgical lamp, an operating room light, an oxygenizer, anesthetic equipment, decontamination equipment, an examination lamp, an incubator, an autoclave, a heart-lung machine, a patient monitor, a patient supervision system, a transport stretcher, an ultrasonic atomizer, or an X-ray apparatus.

Application or introduction of an indicator element such as a photochromic surface may be performed via any of a variety of methods. Optionally, an indicator element such as a photochromic surface is introduced during formation of the indicator element and may be fixedly attached or releasably coupled to an article or surface to be sterilized or disinfected. Optionally, an indicator element such as a photochromic surface is applied to an existing article or surface to be sterilized or disinfected. Optionally, the indicator element comprises an adhesive strip, a pod, a panel, tubing, woven fabric, nonwoven fabric, paper, wrapper, and/or a surface coating that may be coupled to the article to be sterilized or disinfected.

Optionally, the exposure of an article, area, or surface to electromagnetic energy (e.g. UV-C light) for sterilization or disinfection occurs in a medical care facility such as a hospital, a nursing home, an extended care facility, a hospice care facility, or a mobile medical care facility. Optionally, exposure occurs in an operating room. In still other options, exposure occurs in a medical treatment room or a patient room. In still other options, exposure occurs following the discharge or transfer of a patient. In other options, exposure of an article, area or surface to be disinfected or sterilized occurs in or on a cruise ship, school, day care center, camps, restaurant, hotel, or a military setting. It is specifically contemplated that any setting or environment that would benefit from sterilization or disinfection or contains an article, area, or surface that would benefit from sterilization or disinfection may be appropriate for some options.

Pathogens

The methods described herein may be used to detect the sterilization or disinfection of articles, areas, and surfaces by the inactivation of pathogens, among other uses. The following is a non-limiting, exemplary list of pathogens that can be inactivated according to some options of the present disclosure. This list also provides representative diseases caused by the pathogens: bacteriophage (E. coli), Human immunodeficiency virus (HIV), infectious hepatitis, influenza (flu), poliovirus-poliomyelitis, tobacco mosaic virus, rotovirus, S. bacillus anthracis (anthrax), Bacillus magaterium sp. (spores), Bacillus magaterium sp. (veg), Bacillus paratyphusus, Bacillus subtilus (spores), Bacillus subtilis, Clostridium tetani (tetanus/lockjaw), Clostridium difficile, Corynebacterium diphtheriae (diphtheria), Eberthella typosa, Escherichia coli, Leptospira canicoal-infections (e.g., jaundice), methicillin-resistant Staphylococcus aureus (MRSA), Micrococcus candidus, Micrococcus spheroids, Mycobacterium tuberculosis (tuberculosis), Neisseria catarrhalis, Pseudomonas aeruginosa, Pseudomonas fluorescens, Salmonella enteritidis, Salmonella paratyphi (enteic fever), Salmonella typhosa (typhoid fever), Salmonella typhimurium, Sarcina lutea, Serratia marcescens, Shigella dysenteriae (dysentery), Shigella flexneri-(dysentery), Shigella paradysenteriae, Spirillum rubrum, Staphylococcus albus (staph infection), Staphylococcus aureus (staph infection), Streptococcus hemolyticus, Streptococcus lactis, Streptococcus viridians, Vibrio comma (cholera), and mold spores including but not limited to Aspergillius flavis, Aspergillius glaucus, Aspergillius niger, Mucor racemosus a, Mucor racemosus b, Oospora lactis, Penicillium expansum, Penicillium roqueforti, Penicillium digitatum, and Rhisophus nigricans.

Inactivation of pathogens can be readily determined by any of the provided methods described herein. In addition to observation or detection of visual and/or textural changes to an indicator element such as a photochromic surface, other methods known in the art can also be used to verify the effect of exposure to electromagnetic energy on a surface. For example, environmental cultures may be used to determine the effect of, e.g., UV radiation on microbial levels. Exemplary methods are described in: Anderson et al., Infection Control and Hospital Epidemiology, 34(5):466-471, 2013.

Unless otherwise stated, all literature citations made in this specification are herein incorporated by reference in their entirety as if the full disclosure therein were specifically recited herein.

### Examples

This disclosure illustrates optional indicator elements including optional photochromic surfaces, and optional methods for using the indicator elements and the photochromic responses to detect the achievement of desired levels of sterilization or disinfection of an article.

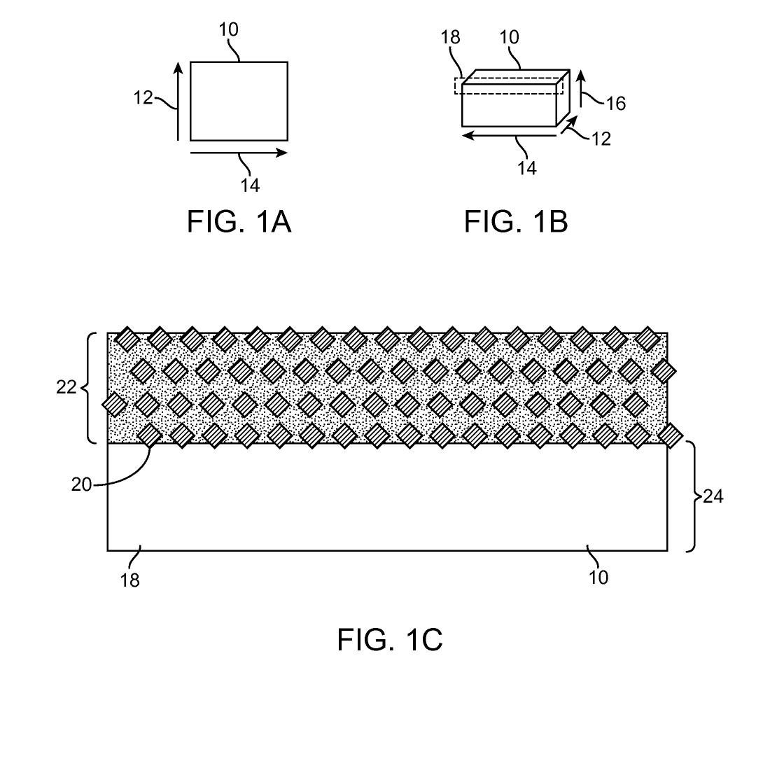

In the example in FIGS. 1A-1C, an indicator element, with a photochromic surface or material is formulated into a rectangular shape 10 having an optional width 14 approximately 1 cm wide, an optional length 12 approximately by 1 cm long, and an optional height 16 approximately 0.5 cm in height. One of skill in the art will appreciate that these dimensions are not intended to be limiting and that any length, width, or height may be used. In this example, the indicator element includes a photochromic surface which comprises a photochromic molecule embedded in a substrate. The substrate may be a layer of polymer as will be described elsewhere. In this example, the photochromic surface is optionally approximately 100 μm thick but any thickness may be used depending on the application.

Exposure to electromagnetic energy (e.g., UV-C radiation), can be used to provide visual feedback of sterilization or disinfection (e.g., sterilization or disinfection of at least one surface of an article). The indicator can be either temporarily or permanently attached to a critical surface or article to be sterilized or disinfected in, for example, a medical or clinical environment. Representative surfaces or articles being sterilized or disinfected include, but are not limited to, a patient bed, an operating table, a tray table, an over-the-bed table, a wheelchair, an electrocardiogram machine, a nursing station computer, and/or the walls of a patient room. Any of the other representative surfaces or articles disclosed herein are also applicable.

FIG. 1A illustrates a top view of the indicator element 10 and FIG. 1B is a side view of the indicator element 10. FIG. 1C is a magnified side view of a section 18 of the indictor element seen in FIG. 1B. The layer 22 of the indicator element 10 having the photochromic material 20 is optionally about 100 microns thick, although any thickness may be applicable. The photochromic material is optionally uniformly distributed in the upper layer 22 of the indicator element 10, and a lower layer 24 may be free of the photochromic material. Optionally, the photochromic material may be disposed in both the upper layer 22 or the lower layer 24, and it may be dispersed uniformly or non-uniformly in either layer. The photochromic material may be any of the photochromic materials disclosed herein or known in the art. The layers may be made from any of the materials described herein. For example, the lower layer 24 may be polyethylene, and the upper layer may be a photochromic material dispersed in a layer of polyethylene. One of skill in the art will appreciate that any photochrome disclosed herein or known in the art may be used, and similarly any polymer disclosed herein or known in the art may also be used.

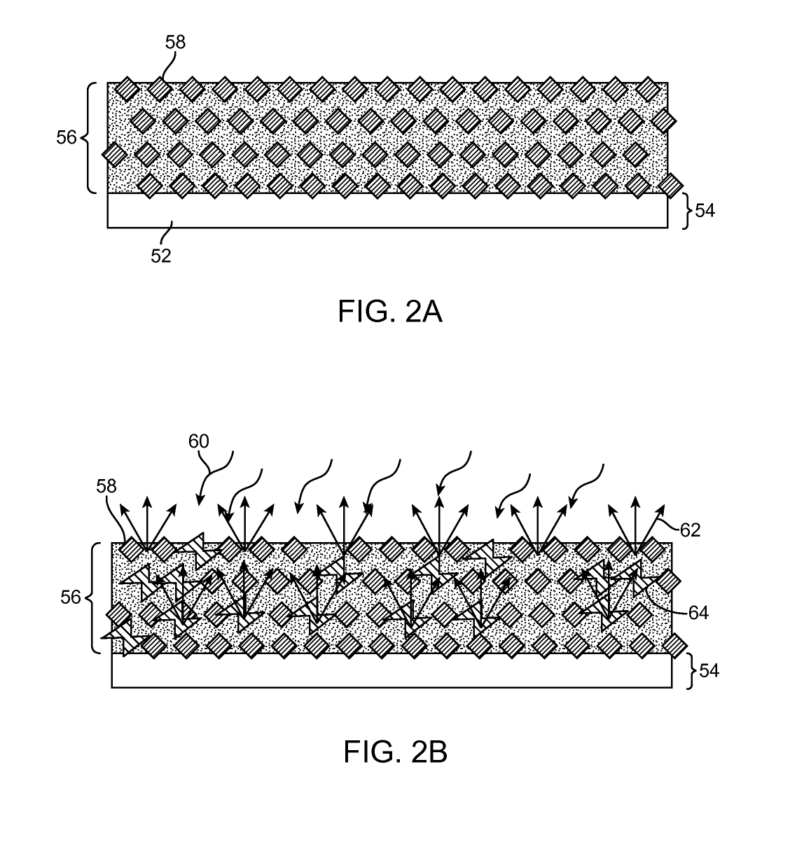

As shown in FIGS. 2A-2B, in this example, upon stimulation of the photochromic surface with UV-C energy, the nature and/or form of the photochromic molecule changes. Specifically, FIG. 2A shows a side view of indicator element 52 with photochromic molecules 58 in the substrate upper layer 56 preferably having a thickness of about 100 microns prior to stimulation with UV-C energy. The upper layer 56 may be formed by evenly dispersing any of the photochromic materials described herein in a polymer layer such as polyethylene. The optional lower layer 54 may also be polyethylene and may be free of photochromeric materials. FIG. 2B shows the photochromic molecules 58 in the substrate during stimulation with UV-C energy. FIG. 2B shows that during stimulation with UV-C energy 60, the stimulated photochromic molecules 64 emit visible light 62 and/or reflect a range of visible light that is different from that reflected by the unstimulated photochromic molecules in FIG. 2A. Exemplary suitable sources of UV-C radiation include those described in International Application No. PCT/US2013/0767173, filed Dec. 19, 2013, now published as WO 2014/100493.

In response to the UV-C energy, the photochromic molecules are then activated as indicated, in this example, by a visual (i.e. color) change in the indicator element. The activation may be reversible or irreversible, as desired for the particular application. In the case of a hospital environment, such as a hospital bed or a surgical instrument, reversible activation may typically be desired to allow for repeated assessments of sterilization or disinfection. Color change may be any number of colors and optionally is from clear to a purple color.

Once the desired level of sterilization or disinfection has been achieved, as indicated by the color change shown in the indicator element, the operator may then terminate the exposure to electromagnetic energy or determine that further exposure is required. One of skill in the art will appreciate that the level of exposure indicates level of disinfection or sterilization. Surpassing 350,000 μW-sec/cm² may kill all classes of organisms on earth. The level of exposure is therefore dictated by the organisms of interest. In the healthcare setting the level of exposure to cover the organisms of interest may be approximately 60,000 μW-sec/cm² or less.

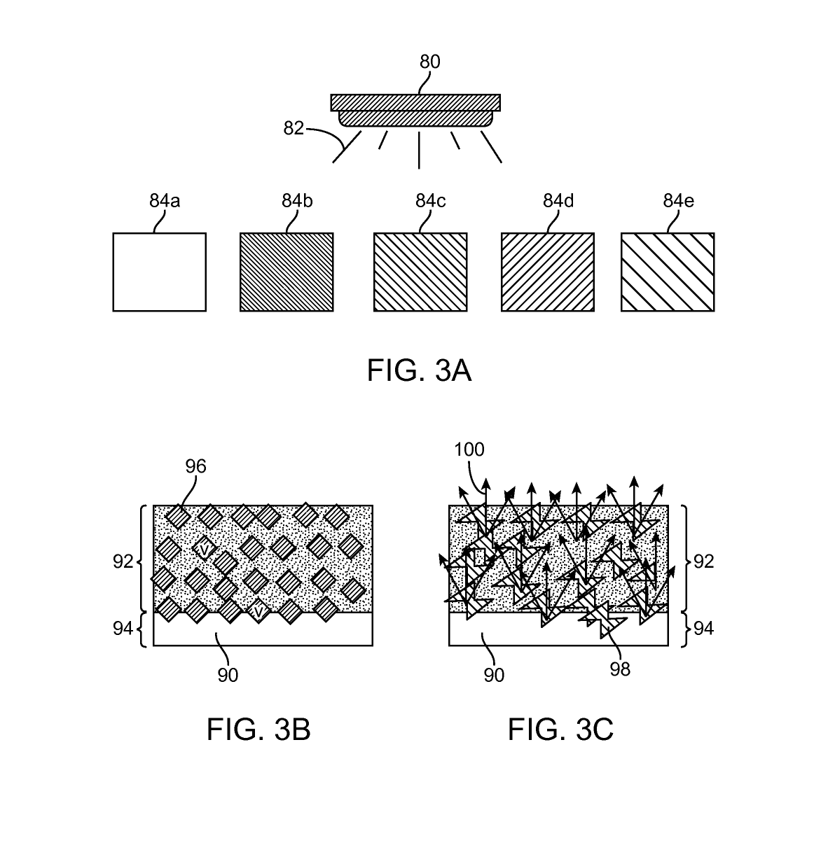

Optionally, the color change shown may be gradual and/or variable. For example, FIG. 3A shows a UV-C source 80 irradiating several different indicator elements independently with UV-C 82. The source provides 1500 μW-sec/cm², and each indicator element is optionally has a surface area of 1 square centimeter. The first indicator element 84a is illuminated for 0 seconds, and therefore receives 0 μW-sec/cm² and indicates a 0% microbial kill efficacy, as evidences by its unchanged color from its natural state (here, optionally clear). Indicator element 84b is illuminated for approximately 30 seconds and receives 45,000 μW-sec/cm² and has a 25% microbial kill efficacy as indicated by its slight color change. Indicator element 84c receives 60 seconds of irradiation and receives 90,000 μW-sec/cm² for a microbial kill efficacy of 50% as indicated by its color change which is darker and deeper in color than the previous indicator element. Indicator 84d changes color even more after being irradiated for 90 seconds to receive 135,000 μW-sec/cm² for a microbial kill efficacy of 75% as indicated by its even further darker and deeper color change, and indicator 84e is the darkest color after irradiation for 120 seconds to receive 180,000 μW-sec/cm² and provides 100% microbial kill efficacy as shown by its color. Thus, the color change of the indicator element may be calibrated to the degree of sterilization or disinfection desired for the specific application. Greater color change in this example indicates greater sterilization or disinfection and hence monitoring of the indicator color may be used to control exposure and duration of the energy to the surface or article being sterilized or disinfected as well as provide a reliable feedback loop to the user of the UV energy source. Continuous exposure of an indicator element such as a photochromic surface (and the photochromic molecules therein) to UV-C energy for 0, 30, 60, 90, or 120 seconds results in progressive changing of color of the article. In this example, the color change is a progressive darkening, as the length of time during which the photochromic surface is exposed to UV-C energy increases.

FIG. 3B shows a schematic representation of the change occurring to the indicator element 90 having photochromic molecules 96 in the photochromic layer 92 during exposure to UV-C energy. A lower or bottom layer 94 may also be included in the indicator element and that layer may not contain any photochromic material. Specifically, FIG. 3B shows the article at time 0 seconds and FIG. 3C after 120 seconds as defined in the timeline of FIG. 3A. After 120 seconds of exposure to 180,000 μW-sec/cm² UV-C energy, the activated photochromic materials 98 emit visible light 100. FIGS. 3A-3C show a graphical representation of a change in molecular form that may occur in a photochromic surface during exposure to UV-C energy.

Blockers:

Optionally, any of the devices or methods described herein may also include blockers which block out other wavelengths of electromagnetic energy which may interfere with the photochromic material. For example, preferably, blockers include materials which block ultraviolet A (UV-A) and/or ultraviolet B (UV-B) radiation. Unwanted UV-A and UV-B can penetrate through the earth's atmosphere and can cause an unwanted change in color or texture to the indicator element which may comprise photochromic surfaces or materials, therefore it would be desirable to provide a blocker that prevents the UV-A or UV-B from irradiating the indicator element, thus preferably the indicator element indicates only exposure to UV-C. Since most of the UV-C is shielded by the earth's atmosphere, UV-C substantially does not penetrate through the atmosphere and hence any UV-C exposed to the indicator element would come from the sterilization or disinfection source, allowing more precise control of the radiation delivered. Thus, optionally the color change indicated by the photochromic material is inactive when exposed to UV-A or UV-B. Optionally, the blocker may be a benzophenone with any concentration, although concentrations ranging from about 5% to about 50% may be used. Other blockers are disclosed herein, and are also known in the art.

### Additional Examples

The indicator element may be formed from one or more layers of material that are coupled together. A polymeric layer of material (e.g. polyethylene) will prevent some UV-C from passing therethrough depending on its thickness. Transmission is roughly inversely linear with the thickness of the polymer. Thus, the thicker the polymer layer, the less UV-C passes through the layer. Therefore, the thickness cannot be so large as to prevent exposure of an indicator element such as a photochromic material dispersed in the polymer layer of the indicator element.

Table 2 below summarizes an experimental setup which demonstrates the effect that the polymeric film or layer has on transmission of UV light through the film and measured by a sensor (UV meter) behind the film or layer.

TABLE 2

| | Reading #1 | Reading #2 | Reading #3 | Average |
|---|---|---|---|---|
| Film Thickness | μW/cm² | μW/cm² | μW/cm² | μW/cm² |
| Control, no film | 611 | 601 | 596 | 603 |
| 2 mil PE film | 505 | 495 | 490 | 497 |
| 4 mil PE film | 410 | 402 | 403 | 405 |
| 6 mil PE film | 389 | 392 | 393 | 391 |
| 8 mil PE film | 332 | 332 | — | 332 |
| 10 mil PE film | 270 | 270 | 271 | 270 |

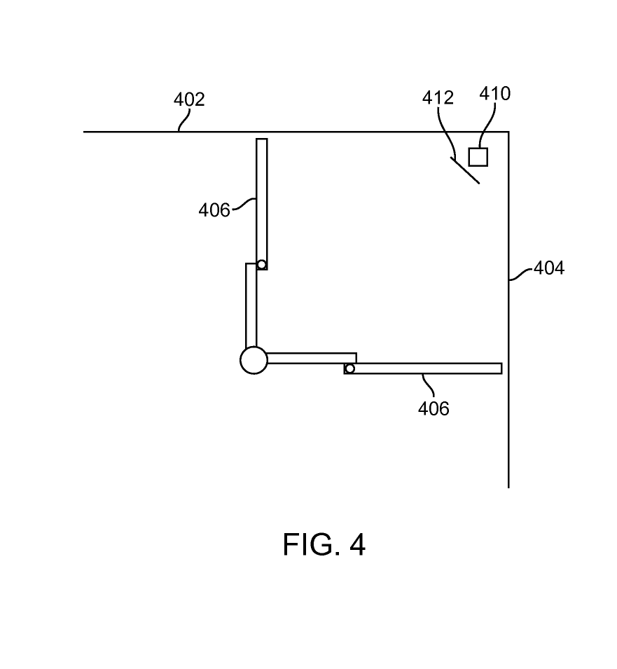

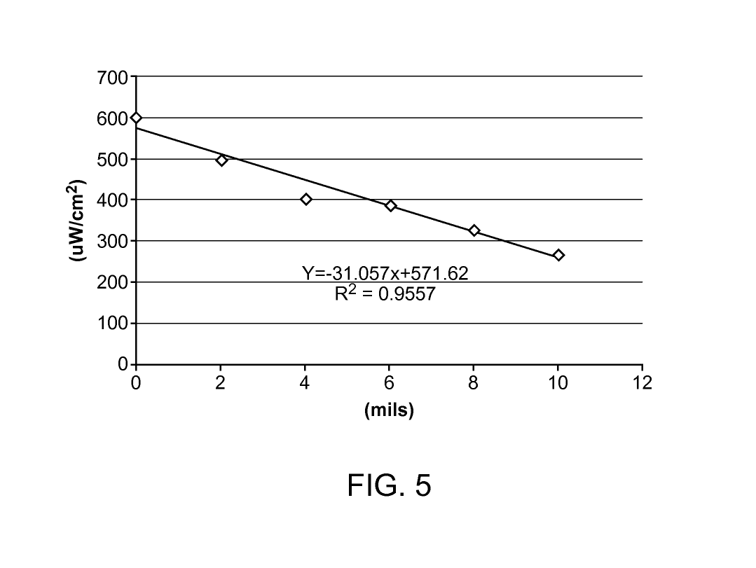

The experimental setup is schematically illustrated in FIG. 4 in which panels 406 of a UV-C light source such as that described in International Application No. PCT/US2013/076717, filed Dec. 19, 2013, now published as WO 2014/100493, are unfolded and extended in a room having adjacent corner walls 402 that are approximately 10 feet long. Varying thickness blank low density polyethylene (PE) films without any blockers, or without photochromeric materials are placed in front of a UV meter 410 and the amount of UV light passing through the film 412 is measured by the UV meter 410 and recorded in Table 2 above. The data is plotted in FIG. 5 which shows a fairly linear relationship between UV-C transmission through the film and film thickness. In this experiment, the correlation coefficient was approximately 0.96. Thus it is clear that the thicker the film layer is, the less UVC-C passes through the film so transmission is inversely proportional to film thickness.

Using a similar setup as illustrated in FIG. 4, the transmission testing was repeated but this time with a varying amount of UV blocker dispersed in the film. In this experiment, benzophenone blocker was dispersed in either 5% or 50% concentrations (by weight) in the polyethylene films and UV transmission was measured. Table 3 below summarizes the data.

TABLE 3

| | Reading #1 | Reading #2 | Reading #3 | Average |
|---|---|---|---|---|
| Film | μW/cm² | μW/cm² | μW/cm² | μW/cm² |
| Control, no film | 630 | 638 | 640 | 636 |
| PE film + 5% | 5 | 5 | 5 | 5 |
| benzophenone | | | | |
| PE film + 50% | 4 | 4 | 4 | 4 |
| benzophenone | | | | |

Thus, the blocker proved to be effective in eliminating 99% of the UV light passing through the polyethylene film. Because photochromic molecules tend to be broad in absorbance, a blocker may be useful to preventing certain wavelengths from passing through the film and exposing the indicator element, such as UV-A and UV-B so that the indicator only works in the presence of UV-C. Therefore, any blocker may be selected and used in order to block selected wavelengths of light or energy from the indicator element and they may be used in the appropriate concentration. Preferably the blocker may be selected to reduce or eliminate UV-A and/or UV-B exposure to the indicator element.

The layers (also referred to as films) may be prepared by methods known in the art. Optionally, a layer may be prepared by dissolving polyethylene in a solvent such as hot xylene at a concentration of about 0.5% to about 5%, and optionally from about 2% to about 3%. The resulting solution is filtered to remove undissolved polymer. Photochromeric materials and/or blockers are dispersed preferably evenly throughout the polymer solution. A film or layer is then cast by pouring the solution onto a flat surface and allowed to evaporate resulting in a polymer film. The polymer film may be polymer only, polymer with blocker, polymer with photochromeric material, or combinations thereof. The various film layers may then be put together in any desired configuration.

The films may be formed from any number of materials. Optionally, the films are formed from melt-processable semicrystalline thermoplastic polymer resin for example a polyethylene resin of low density, medium density, linear low density, high density, or ultra high molecular weight. Alternatively, semi-crystalline materials such as Nylon 6, Nylon 66, acetal, etc. may be used.

Optionally, melt-processable amorphous thermoplastic resins such as acrylonitrile butadiene styrene (ABS), acrylic, polycarbonate, and the like may be used to form the films. Other material options would be thermoset resins such as epoxy and or thermoplastic elastomers. Optionally, the film is formed with a homopolymer, that is one homogenous polymer material. Alternatively blends of two or more polymers, such as polycarbonate and ABS are an alternative embodiment. Furthermore, co-polymers in which multiple polymers are polymerized together are also optional.

Sample Film Preparation:

Various films were prepared using the following methods which are not intended to be limiting. The sample indicator elements were constructed in a film configuration in order to demonstrate enablement but are not intended to be limiting.

1. Dissolve Lyondell Basell Lupolen 2420F low density polyethylene (LDPE) by stirring in hot xylene (Sigma Aldrich reagent grade) (90-110° C.) on a conventional hot plate with a mechanical stir bar.

a. The resultant solution was between 2% and 3% by weight in xylene.

b. Initial concept development was completed with an LDPE from Dow Chemical (Grade 772).

c. The Lupolen 2420F is slightly higher density material with a higher melting temperature (Tₘ) than the Dow 772.

d. Matching viscosity and evaporation rate of the materials may help to minimize mud-cracking.

2. After the majority of the LDPE has dissolved into the solvent solution, the resultant solution was filtered to remove undissolved polymer.

a. The resultant fluid yielded a clear solution of ˜2% LDPE in xylene.

3. Several films were produced with varying additives. In the event a film was produced with an additive, the additive was included at this stage.

a. The resultant blend would be mixed for a minimum of five minutes using a mechanical stir bar on a hot plate at 90-110° C.

b. In general, the photochromic molecules and UV blockers utilized in this program were thermally stable at 90-110° C.

4. Films were initially cast by pouring the dilute polymer solution onto flat glass substrates.

a. The solvent evaporates as a function of time.

b. The resultant film is then separated from the glass substrate.

c. The concentration of polymer within the solvent solution was varied from 0.5% to 5% to observe the effect on mud cracking.

d. The final film casting process utilized a heated a flat metal substrate (at 120° C.).
i. A Fluorofab release film was applied to the substrate.
ii. A 0.050″ drawdown bar was utilized to create a controlled thickness.
iii. Xylene evaporated as a function of time.
iv. Processing challenges may still exist; if the wet film temperature dropped below 100° C. before the majority of the xylene was evaporated (i.e. >90% evaporated), mud-cracking occurs as the LDPE crystallizes.
v. This modified process yields films that are ˜0.001″ thick.

e. The final films cast include:
i. LDPE
ii. LDPE+1% Cinnibar Reversacol photochromic molecule
iii. LDPE+2% UV-A/UV-B blocker (benzophenone)

Another optional method for preparing films is outlined below.

1. Dissolve Dow LDPE 772 low density polyethylene (LDPE) by stirring in hot xylene (Sigma Aldrich reagent grade) (90-110° C.) on a conventional hot plate with a mechanical stir bar.

2. Several films were produced with varying additives. In the event a film was produced with an additive, the additive was included at this stage.

a. The resultant blend would be mixed for a minimum of five minutes using a mechanical stir bar on a hot plate at 90-110° C.

b. In general, the photochromic molecules and UV blockers utilized in this program were thermally stable at 90-110° C.

3. Films were cast by pouring onto a flat glass dish on a hot plate at ˜140° C.

4. Cover dishes loosely to slow xylene evaporation and slightly improve surface finish. Allow xylene to evaporate.

5. This process yielded films with a thickness of approximately 0.005″ to 0.015″.

6. The initial cast films included:

a. LDPE

b. LDPE+2 wt % solids Cinnibar Reversacol photochromic molecule

c. LDPE+5 wt % solids benzophenone blocker

d. LDPE+50 wt % solids benzophenone blocker
i. Poor miscibility at this high of concentration, so actual concentration was likely 20-30%.
ii. Used benzotriazole as a blocker.

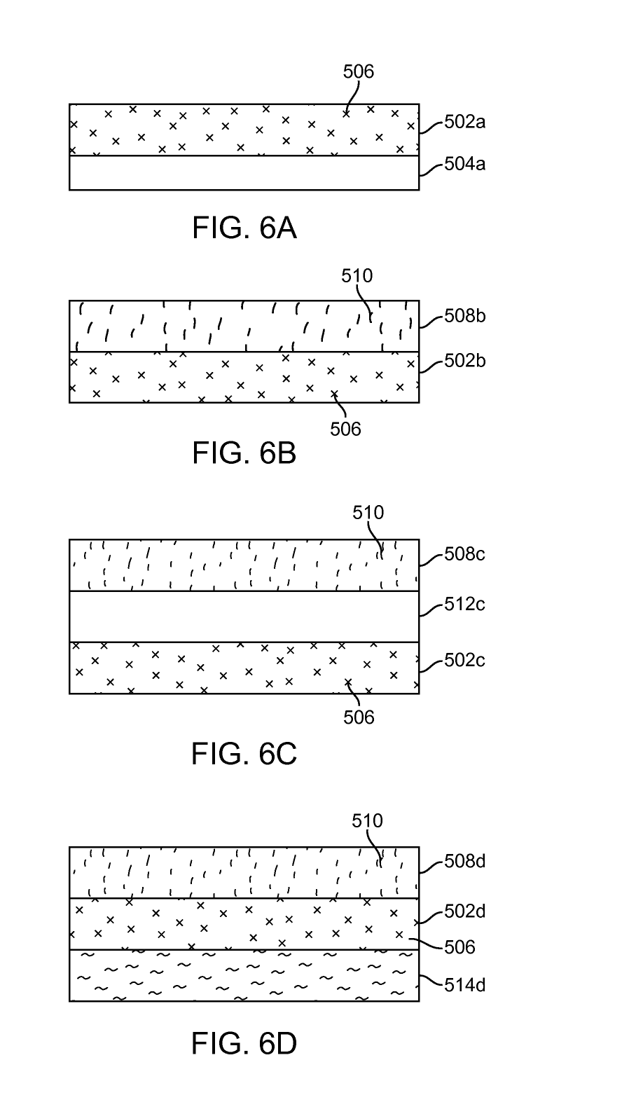

FIGS. 5A-5D illustrate several optional constructs of an indicator element formed from one or more polymer layers. For example, FIG. 6A shows an upper layer 502a and a lower layer 504a. The upper layer may contain photochromic materials 506 dispersed (evenly or randomly) in a polymer film such as polyethylene. The second layer 504a may be a polythene film.

FIG. 6B shows an upper layer 508b and a lower layer 502b. The upper layer 508b may include any of the blockers 510 disclosed herein which prevent UV-A and UV-B from passing through the indicator and they may be dispersed evenly or randomly in a polymer layer such as in polyethylene. The lower layer includes the photochromic material 506 which is preferably dispersed evenly or randomly in a polymer layer such as polyethylene.

FIG. 6C shows an upper layer 508c, a middle layer 512c, and a lower layer 502c. The upper layer 508c may include blockers 510 to prevent UV-A or UV-B from passing through the indicator and they may be dispersed evenly or randomly in a polymer layer such as in polyethylene. The middle layer 512c may be a layer of polyethylene or any other polymer. The middle layer thickness may be adjusted in order to attenuate the amount of UV-C that passes through the middle layer and that irradiates the bottom layer. The bottom layer may be a polyethylene layer with photochromic materials 506 dispersed evenly or randomly therein.

FIG. 6D shows an upper layer 508d, a middle layer 502d, and a bottom layer 514d. The upper layer 508d may include blockers 510 to prevent UV-A or UV-B from passing through the indicator and they may be dispersed evenly or randomly in a polymer layer such as in polyethylene. The middle layer 502d may be a polyethylene layer with photochromic materials 506 dispersed evenly or randomly therein. The bottom layer 514d may be any substrate such as a magnet to allow coupling of the device with an article to be sterilized or disinfected or any work surface, or the bottom layer 514d may be an adhesive, Velcro or other hook and loop fasteners, or any other means for fastening the indicator to the work surface either releasably or fixedly. Any of the photochromic materials, or any of the blockers or film materials may be used in any of the examples. Also, the layers are not limited to polymer layers. The layers may be formed from any number of materials such as glass, metal, cardboard, composites, ceramics, paper, etc. Any permutation or combination of the materials and layers may be made to provide an indicator element.

Optionally, the photochromic molecule may be chemically grafted onto a polymer backbone and therefore the photochromic material need not be added separately to the polymer film layer.

As discussed previously, photochromic molecules are molecules in which light-induced reversible change of color are possible. The most prevalent organic photochromic systems are unimolecular reactions in which the base material form is colorless (or near colorless) and upon exposure become colored. Generally, the colored state fades thermally back to the original state—or—via exposure to visible light. Photochromic molecules consist of complex molecular structures which incorporate aromatic complexes. Optionally, the device incorporates a Spiropyran (more commonly referred to as a “Pyran”. Other options include Spirooxazines (“Oxazine”), Chromenes, Fulgides/Fulgimides, Diarylethenes, Azo Compounds, Spirodihydroindolzines, Polycyclic Aromatic Compounds, Anils, Polycyclic Quinones, Triarylmethanes, Viologens, and Perimidine-spirocycyclohexadienones. Alternatively, this option includes functional derivatives of these in which molecular modifications of such molecules have been made to incorporate physical or chemical bonding of the molecules to fillers, additives, or the polymer itself. Optionally, indicators include on or about 0.1% by weight to on or about 5% of the photochromic material. Optionally, the indicators include 0.1% to 15% or optionally 0.1% to 25% photochromeric material. Optionally, only one photochromeric material is used, but optionally a plurality of photochromeric materials may be used.

Optionally, the photochromeric material may be a 1%-2% concentration of Cinnibar Reversacol, and the blocker may be benzophenone or benzotriazole. In addition to casting films, the films may be injection molded. Multiple films may be stacked on top of one another to form the complete assembly. For example, a two shot injection molding process may be used to first form one layer and then form a second layer around the first layer. Other manufacturing processes which may optionally be used to produce the device include other injection molding techniques such as over molding, co-injection molding such as the Kortec process, as well as extrusion, co-extrusion, thermoforming, compression molding, transfer molding, film casting, sheet extrusion, compression, thermoforming, 3-D printing, electrospinning, or other similar melt-processing technique for thermoplastic polymers or thermoset polymers.

The blockers may be any blocker known in the art including benzophenone or benzotriazole. Percentages may range from about 0.01% to 25% by weight, and preferably about 0.01% to 5%, and more preferably about 0.01% to 0.5%.

Optionally, secondary additives may also be added to any layer of the film or otherwise to the indicator device. For example, phosphorous or amine based heat stabilizers such Irganox® and Irgafos® may be added. Also optionally, anti-slip and processing aids such as mineral oil may also be added to any of the layers or to the indicator device.

Similar provided indicator elements and/or methods may be used with any of a variety of other options, including medical devices or structural or other articles or surfaces to be sterilized or disinfected and that incorporates an indicator element such as a photochromic molecule or compound on and/or into at least a portion of at least one surface. Optionally, the devices and methods described herein may be combined with a tape or an adhesive that allows the indicator element to be attached to the article or surface undergoing sterilization or disinfection.

Thus the indicator elements here, including photochromic materials may be used to quantify UV-C dosage based on the amount of energy that penetrates through different layers and compositions of the indicator element.

The indicator element optionally may exhibit a visual or textural change with a gradient or immediately upon exposure the electromagnetic energy. Thus, a larger visual or textural change may be indicative of greater exposure to electromagnetic energy, and a lower visual or texture change may be indicative of less exposure to the energy. Examples of such visual or textural changes optionally include an optical change of color, contrast, shading or blending of the article or indicator element when the presence of UV-C energy, and the increase or decrease of excitation may be governed by variables such as intensity or dosage or proximity or time or temperature or energy placed onto the indicator element. Alternatively, the indicator element may simply indicate a color change which occurs immediately upon exposure to electromagnetic energy indicating UV-C excitation and thus that sterilization has begun. A plurality of photochromic molecules may be contained within the indicator element to provide visual—such as color change—or—geometrical patterns to indicate elapsed time, absorbed dose, or dose rate or the like.

While this disclosure has mainly focused on the use of photochromic materials as the indicator element, this is not intended to be limiting. Other indicator materials may also be used. For example, a light emitting diode (LED) may also be used as an indicator element.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of sterilizing or disinfecting an article, said method comprising:
providing an indicator element adjacent the article to be sterilized or disinfected, wherein the indicator element reacts to and indicates exposure to electromagnetic energy;
exposing the indicator element and article to electromagnetic energy having a wavelength between about 100 nm and about 280 nm, wherein the exposure results in one or more visual and/or textural changes to the indicator element;
detecting the one or more visual and/or textural changes of the indicator element; and
terminating the electromagnetic energy exposure after detecting the visual and/or textural changes consistent with sterilization or disinfection of the article adjacent the indicator element.

2. The method of claim 1, wherein the indicator element comprises a photochromic surface or material.

3. The method of claim 1, wherein the visual and/or textural changes are irreversible.

4. The method of claim 1, wherein the visual and/or textural changes are reversible.

5. The method of claim 1, wherein the electromagnetic energy comprises light with a wavelength between about 100 nm to about 120 nm, about 120 nm to about 140 nm, about 140 nm to about 160 nm, about 160 nm to about 180 nm, about 180 nm to about 200 nm, about 200 nm to about 220 nm, about 220 nm to about 240 nm, about 240 nm to about 260 nm, or about 260 nm to about 280 nm.

6. The method of claim 1, wherein the electromagnetic energy comprises light with a wavelength between about 240 nm to about 260 nm.

7. The method of claim 6, wherein the electromagnetic energy comprises light with a wavelength that is about 254 nm.

8. The method of claim 1, wherein the electromagnetic energy results from a source providing an output of about 100 watts to about 1000 watts.

9. The method of claim 8, wherein the electromagnetic energy results from a source providing an output of about 800 watts.

10. The method of claim 1, wherein the article or indicator element is exposed to a dose of electromagnetic energy between about 10,000 to about 60,000 μW-sec/cm².

11. The method of claim 1, wherein the article or indicator element is exposed to electromagnetic energy for between about 0.01 seconds to about 120 seconds.

12. The method of claim 11, wherein exposing comprises exposing the article or indicator element to the electromagnetic energy for between about 10 seconds to about 60 seconds.

13. The method of claim 1, further comprising positioning the article and indicator element within about 0.5 to about 12 feet from a source of the electromagnetic energy.

14. The method of claim 1, wherein the indicator element comprises an adhesive strip, a pod structure, a panel, tubing, woven fabric, nonwoven fabric, paper, wrapper, or a surface coating.

15. The method of claim 1, wherein the article is selected from a piece of furniture, a computer, a wall, a counter, a piece of diagnostic equipment, a piece of medical equipment, a piece of laboratory equipment, a railing, a sink, a toilet, a shower, a trash receptacle, a surgical instrument, a telephone, a remote control, a light switch, bedding, and an electrocardiograph.

16. The method of claim 1, wherein the article is selected from: a door handle, a hospital bed, a patient bed, an operating table, a medical tray, a med cart, a wound cart, a wheelchair, a food tray, a perfusion pump, endoscopy equipment, ventilator equipment, a heart-lung bypass machine, a medical light, a medical vacuum system, a surgical lamp, an operating room light, an operating table, an oxygenizer, anesthetic equipment, decontamination equipment, an examination chair, an examination lamp, an examination table, an incubator, an autoclave, a heart-lung machine, a patient monitor, a patient supervision system, a transport stretcher, an ultrasonic atomizer, an X-ray apparatus, and an electrocardiograph.

17. The method of claim 15, wherein the piece of furniture is a bed, a table, or a chair.

18. The method of claim 1, wherein the exposing occurs in a medical care facility.

19. The method of claim 1, wherein the exposure to the electromagnetic energy results in sterilization or disinfection of at least one surface of the article.

20. The method of claim 1, wherein the sterilization or disinfection also comprises at least one of air disinfection, water disinfection, or surface disinfection.

21. The method of claim 1, wherein the sterilization or disinfection is characterized by a microbial kill rate of at least 85%, at least 88%, at least 90%, at least 91%, at least 92%, at least 93%, at least 94%, at least 95%, at least 96%, at least 97%, at least 98%, at least 99%, at least 99.2%, at least 99.5%, at least 99.9% or at least 99.9999%.

22. The method of claim 1, wherein the sterilization or disinfection results from inactivation of one or more bacteria, molds, protozoa, fungi, viruses, or yeasts, or any combination thereof.

23. The method of claim 2, wherein the photochromic surface comprises a photochrome that is a photochromic monomer, a photochromic oligomer, or a photochromic polymer.

24. The method of claim 2, wherein the photochromic surface comprises a photochrome that is a photochromic small molecule having a molecular weight of less than 1000 Daltons.

25. The method of claim 24, wherein the photochromic small molecule is benzo[3,4]fluoreno[2,1-b]pyran-13-ol, 3,13-dihydro-3,3-bis(4-methoxyphenyl)-6,11,13-trimethyl-,

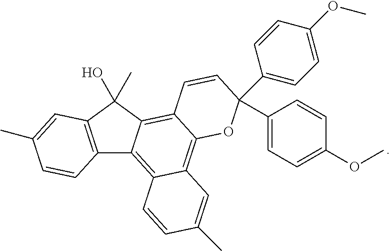

26. The method of claim 23, wherein the photochromic surface comprises an optionally substituted coumarin moiety, an optionally substituted spirooxazine moiety, an optionally substituted naphthoaxazine moiety, an optionally substituted naphthopyran moiety, an optionally substituted phenoxyanthraquinone moiety, an optionally substituted carbazole moiety, or an optionally substituted spiropyran moiety, or combinations thereof.

27. The method of claim 1, wherein the indicator element is releasably coupled to the article.

28. The method of claim 1, further comprising providing a blocking agent and preventing exposure of the indicating element to ultraviolet A and ultraviolet B energy.

29. An article for use in the method of claim 1, wherein the indicator element comprises a photochromic surface.

30. An indicator for indicating sterilization or disinfection of an article, said indictor comprising:
an indicator element configured to react to and indicate exposure to electromagnetic energy, wherein the electromagnetic energy has a wavelength between about 100 nm and about 280 nm, and
wherein the exposure results in one or more visual and/or textural changes to the indicator element; and
a substrate coupled to the indicator element and wherein the substrate is configured to be coupled to an article to be sterilized or disinfected.

31. The indicator of claim 30, wherein the indicator element comprises a photochromic surface or material.

32. The indicator of claim 31, wherein the photochromic surface comprises a photochrome that is a photochromic monomer, a photochromic oligomer, or a photochromic polymer.

33. The indicator of claim 31, wherein the photochromic surface comprises a photochrome that is a photochromic small molecule having a molecular weight of less than 1000 Daltons.

34. The indicator of claim 31, wherein the photochromic small molecule is benzo[3,4]fluoreno[2,1-b]pyran-13-ol, 3,13-dihydro-3,3-bis(4-methoxyphenyl)-6,11,13-trimethyl-,

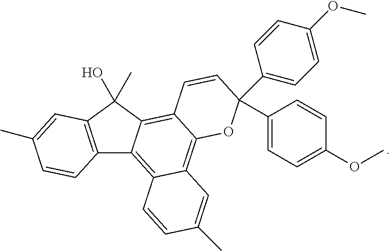

35. The indicator of claim 31, wherein the photochrome comprises an optionally substituted coumarin moiety, an optionally substituted spirooxazine moiety, an optionally substituted naphthoaxazine moiety, an optionally substituted naphthopyran moiety, an optionally substituted phenoxyanthraquinone moiety, an optionally substituted carbazole moiety, or an optionally substituted spiropyran moiety, or combinations thereof.

36. The indicator of claim 30, wherein the indicator element comprises one or more layers of material.

37. The indicator of claim 36, wherein the layer of material comprises a polymer layer.

38. The indicator of claim 30, wherein the substrate is a discrete layer from the indicator element.

39. The indicator of claim 38, wherein the substrate comprises an adhesive for coupling the indicator to the article to be sterilized or disinfected.

40. The indicator of claim 30, wherein the visual and/or textural changes are irreversible.

41. The indicator of claim 30, wherein the visual and/or textural changes are reversible.

42. The indicator of claim 30, wherein the indicator element comprises an adhesive strip, a pod structure, a panel, tubing, woven fabric, nonwoven fabric, paper, wrapper, or a surface coating.

43. The indicator of claim 30, wherein the visual and/or textural changes indicate sterilization or disinfection of the article characterized by a microbial kill efficacy of at least 85%, at least 88%, at least 90%, at least 91%, at least 92%, at least 93%, at least 94%, at least 95%, at least 96%, at least 97%, at least 98%, at least 99%, at least 99.2%, at least 99.5%, at least 99.9% or at least 99.9999%.

44. The indicator of claim 30, further comprising a blocking agent for blocking ultraviolet A and ultraviolet B energy from the indicator element.

45. A system for indicating sterilization or disinfection of an article, said system comprising:
an indicator element configured to react to and indicate exposure to electromagnetic energy, wherein the electromagnetic energy has a wavelength between about 100 nm and about 280 nm, and
wherein the exposure results in one or more visual and/or textural changes to the indicator element; and
a source of the electromagnetic energy for sterilizing or disinfecting the article.

46. The system of claim 45, wherein the indicator element comprises a photochromic surface or material.

47. The system of claim 45, wherein the indicator element is configured in a layer of material.

48. The system of claim 45, wherein the indicator element further comprises a substrate layer, and wherein the substrate layer comprises an adhesive for coupling the indicator to the article to be sterilized or disinfected.

49. The system of claim 45, wherein the visual and/or textural changes are irreversible.

50. The system of claim 45, wherein the visual and/or textural changes are reversible.

51. The system of claim 45, wherein the indicator element comprises an adhesive strip, a pod structure, a panel, tubing, woven fabric, nonwoven fabric, paper, wrapper, or a surface coating.

52. The system of claim 45, wherein the visual and/or textural changes indicate sterilization or disinfection of the article characterized by a microbial kill efficacy of at least 85%, at least 88%, at least 90%, at least 91%, at least 92%, at least 93%, at least 94%, at least 95%, at least 96%, at least 97%, at least 98%, at least 99%, at least 99.2%, at least 99.5%, at least 99.9% or at least 99.9999%.

53. The system of claim 45, further comprising a blocking agent for blocking ultraviolet A and ultraviolet B energy from a photochromic layer in the indicator element.
